(19) United States
(12) Patent Application Publication
(10) Pub. No.: US 2009/0018467 A1
(43) Pub. Date: Jan. 15, 2009

(54) APPARATUS FOR PUNCH BIOPSY

(76) Inventors: KWOK WAI CHIU, Plano, TX (US); Timothy Chiu, Plano, TX (US); William S. Wang, Richardson, TX (US)

Correspondence Address: Dr. Kwok-Wai Chiu, Suite 105, 1220 Coit Road, Plano, TX, 75075, US

(21) Appl. No.: 12/234,696

(22) Filed: Sep. 21, 2008

Related U.S. Application Data
(63) Continuation-in-part of application No. 11/426,313, filed on Jun. 25, 2006, now Pat. No. 7,442,170.

(51) Int. Cl. A61B 10/00

(52) U.S. Cl. 600/562

(57) ABSTRACT

A punch biopsy apparatus for removing all or a portion of a suspect dermal growth. A punch biopsy apparatus has: a hollow cylinder body; a coring blade at the base of the hollow cylinder body; at least one scooping blade pivotally secured by an axle and a pair of pivot seats within the cylinder body, where each scooping blade is semicircular, a longitudinally-moveable plunger secured within the hollow cylinder, where the plunger has at least one plunger leg for contacting each scooping blade, and each plunger leg transfers longitudinal plunger movement to its respective scooping blade, thereby causing rotational scooping-blade movement. This device helps minimize bleeding and minimize damage to the biopsy sample being retrieved.

### CROSS-REFERENCE TO RELATED APPLICATION

This application is a continuation-in-part of prior application Ser. No. 11/426,313 entitled “Apparatus for Punch Biopsy” and filed on Jun. 25, 2006.

### BACKGROUND

1. Technical Field

The present invention relates to a punch biopsy apparatus for use in the surgical removal of all or part of a dermal (or skin) growth. More specifically, an improved punch biopsy apparatus enables biopsy removal with ease, efficiency, and minimal bleeding.

2. Description of Related Art

Skin biopsy is extremely useful in diagnosing potential dermatological disorders. In fact, many incorrect diagnoses occur due to a failure to perform a skin biopsy, or an improperly-executed biopsy. The most common technique used to obtain diagnostic, full-thickness dermatological samples is punch biopsy. The term “punch biopsy” refers to the nature of excising the suspect skin sample for analysis, which is akin to punching holes in a leather belt or punching holes in paper.

In the prior art, punch biopsy is typically performed using a circular blade or cylindrical blade. One specific example prior art device is a trephine, which is a surgical instrument having circular, saw-like edges, often used to cut out disks of bone, but which can also be used to remove cylindrical cores of skin. Such circular or cylindrical blade is rotated against and down through the epidermis first, then through the dermis, and finally into the subcutaneous lipid layer of the skin. When the practitioner is satisfied that the cylindrical blade has reached the appropriate depth, the practitioner then lifts the cored skin sample with either a pair of forceps or the anesthetic needle. While the skin sample is lifted, the sample is removed by cutting through the subcutaneous base with sharp tissue scissors or scalpel.

However, such rough handling of the sample can damage the sample to be tested. Crush artifact is particularly likely when forceps are used to raise the sample. Furthermore, the practitioner may not always be able to cut the base of the sample as low as desirable if the skin is too taught in the biopsy area.

Thus, a need exists for an improved surgical device for use in surgical procedures for removing skin biopsy samples. Such a device should be simple and easy to use even by doctors having minimal training and experience in surgical procedures.

### SUMMARY OF THE INVENTION

According to the present invention, a punch biopsy apparatus has: a hollow cylinder body; a coring blade at the base of the hollow cylinder body; at least one scooping blade pivotally secured by an axle and a pair of pivot seats within the cylinder body, where each scooping blade is semicircular; a longitudinally-moveable plunger secured within the hollow cylinder, where the plunger has at least one plunger leg for contacting each scooping blade, and each plunger leg transfers longitudinal plunger movement to its respective scooping blade, thereby causing rotational scooping-blade movement. This device helps minimize bleeding and minimize damage to the biopsy sample being retrieved.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a side perspective view of a first embodiment of a punch biopsy device according to the present invention with the scooping blade fully retracted;

FIG. 2 is an enlarged perspective view of the first embodiment with the scooping blade and actuating lever apart from the cylinder body;

FIG. 3 is a bottom perspective view of the first embodiment with the scooping blade fully extended;

FIG. 4 is a bottom perspective view of the first embodiment with the scooping blade and actuating lever canted at an appropriate angle for installation and/or removal;

FIG. 5 is a side perspective view of a second, preferred embodiment of a punch biopsy device with a pair of plunger-activated scooping blades in a retracted orientation;

FIG. 6 is a side perspective view of the second embodiment with the scooping blades in a fully-deployed orientation;

FIG. 7 is an elevated bottom view of the second embodiment with the scooping blades in a retracted orientation;

FIG. 8 is similar to FIG. 5 except with the plunger shown in an elevated position relative to the scooping blades;

FIG. 9 is a side perspective view of the second embodiment with the scooping blades in a partially-deployed orientation.

### REFERENCE NUMERALS

112 cylinder body
114 actuating lever
116 pivot seat
118 installation channel
120 scooping blade
122 coring blade
212 cylinder body
214 plunger
216 plunger legs
218 axle
220 scooping blade(s)
222 coring blade
224 plunger guide
226 plunger top
228 finger grip(s)
230 plunger feet
232 pivot seat
238 biopsy sample

### DETAILED DESCRIPTION

While the invention is described below with respect to a preferred embodiment, other embodiments are possible. The concepts disclosed herein apply equally to other instruments for retrieving core samples of other tissues, organs, organic matter, and can be applied to inorganic matter as well, provided that they follow the spirit of the teachings disclosed herein.

Whereas many prior art biopsy devices simply make vertical incisions but do not provide lateral incisions for detaching core samples, the present invention enables one to remove the desired portion of the skin by accomplishing both vertical and lateral incisions without employing a second instrument. Such contoured excision of the excess skin is made possible with the present punch biopsy device as disclosed herein.

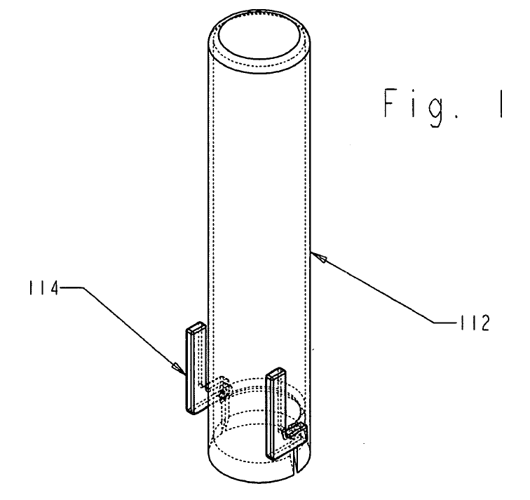

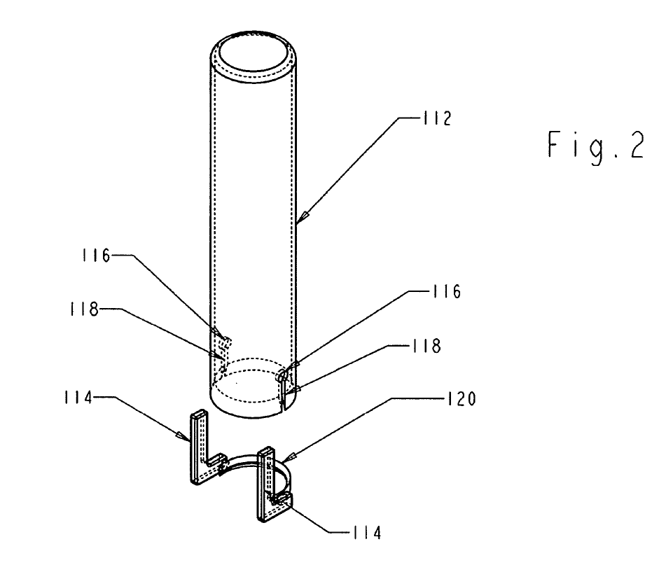

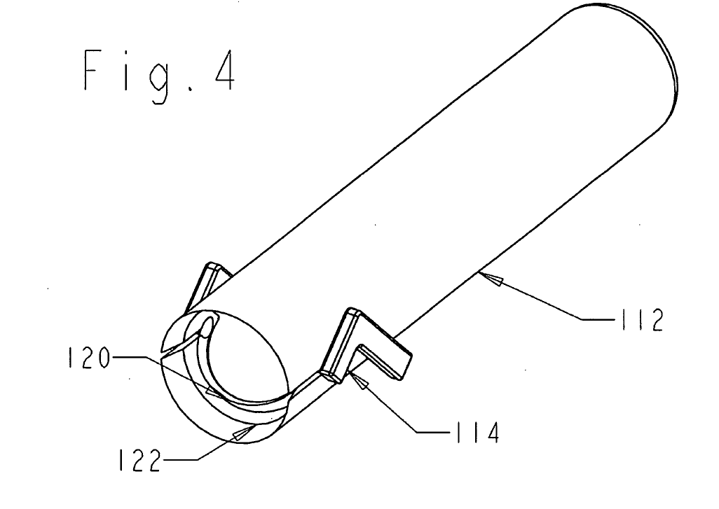

According to the present invention, a first embodiment of a punch biopsy apparatus has: a hollow cylinder body 112; a circular, beveled, coring blade 122 at the base of the hollow cylinder body 112; two circular pivot seats 116 near the base of the cylinder body 112; two longitudinal installation channels 118 at the base of the cylinder body 112 leading from the two circular pivot seats 116 down to the base of the cylinder body 112, essentially dividing the coring blade 122 into halves; and a scooping blade assembly installed through the installation channels 118 and pivotally secured by the pivot seats 116, where the scooping blade assembly comprises a semicircular scooping blade 120 having actuating levers 114 attached at each end via essentially oblong connectors (or pivot joints). This device helps minimize bleeding and minimize damage to the biopsy sample being retrieved. FIGS. 1-4 illustrate this first embodiment.

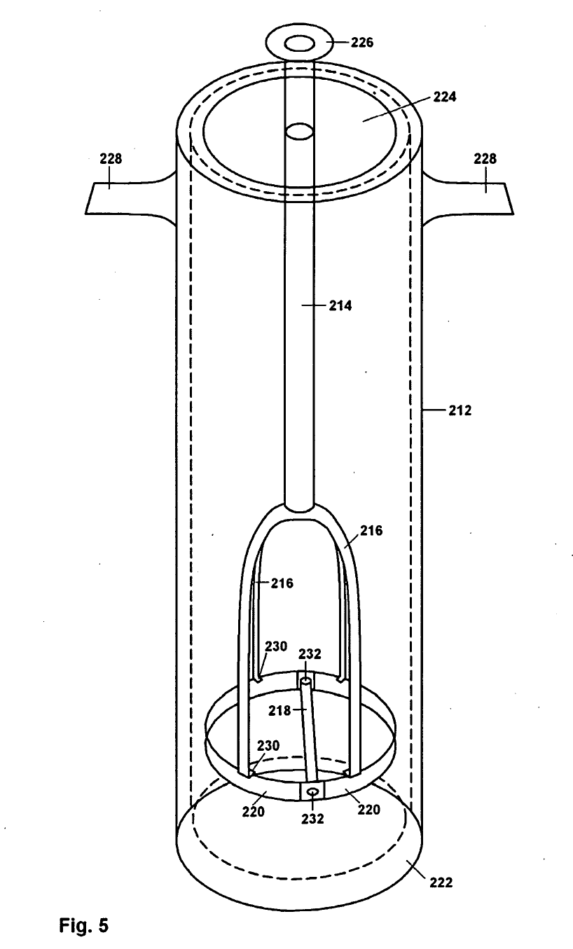

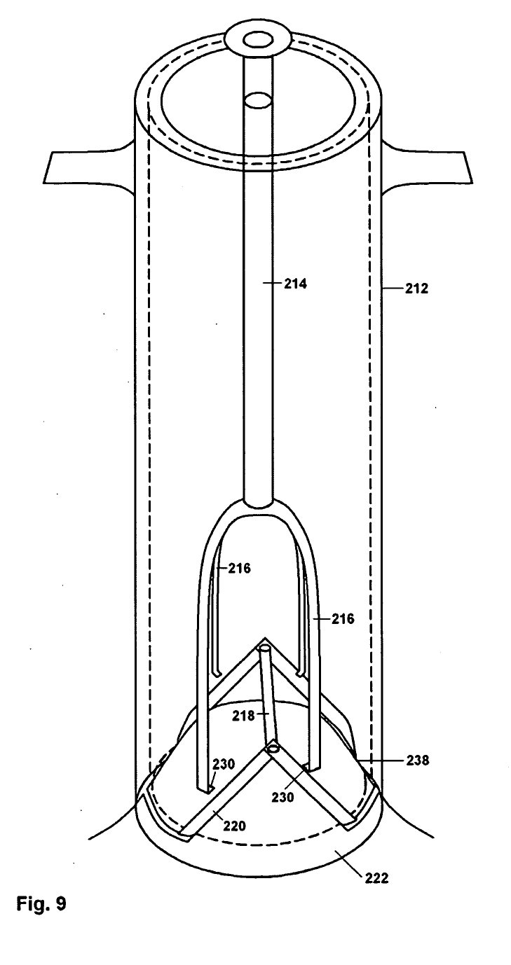

A second, more preferable embodiment of a punch biopsy apparatus has: a hollow cylinder body 212; a coring blade 222 at the base of the hollow cylinder body; at least one scooping blade 220 pivotally secured by an axle 218 and a pair of pivot seats 232 within the cylinder body 212, where each scooping blade 220 is semicircular; a longitudinally-moveable plunger 214 secured within the hollow cylinder body 212, where the plunger 214 has at least one plunger leg 216 for contacting each scooping blade 220, and each plunger leg 216, via longitudinal plunger movement, is capable of applying torque to its respective scooping blade 220, thereby enabling rotational scooping-blade movement about the axle 218. Again, this device helps minimize bleeding and minimize damage to the biopsy sample 238 being retrieved. FIGS. 5-9 illustrate this second embodiment.

The cylinder body 112, 212 in both embodiments can be made of any sturdy, medical-grade material that can be sterilized and is suitable for containing tissue samples without cross-contamination and/or chemical leaching. Example materials include but are not limited to: stainless steel, biocompatible plastics, polymers, composites, ceramics, specialty and exotic metals, alloys, etc. In a preferred embodiment, stainless steel is used for all of the device components, including the cylinder body 112, 212. The inner diameter of the cylinder body 112, 212 is based upon the desired size of the biopsy to be taken. Thus, the inner diameter will typically range from about 2 millimeters (2 mm) to about 5 millimeters (5 mm). Preferably, the inner diameter will be between about 3 mm and about 4 mm. Smaller or larger inner diameters, however, are plausible and should be selected based on the size of the biopsy sample to be taken. The thickness of the cylinder body 112, 212 walls should be thick enough to withstand firm handling and twisting during biopsy procedures but should otherwise be as thin as possible.

The circular, beveled, coring blade 122, 222 in both embodiments is located at the base of the cylinder body 112, 212, and the thickness of the coring blade 122, 222 tapers from its base or upper edge (where it meets the cylinder body 112, 212) down to its sharp cutting edge or leading edge. The coring blade 122, 212 is generally circular except for the pair of installation channels 118 essentially dividing the coring blade 122 into halves (in the first embodiment).

The scooping-blade assembly in the first embodiment comprises a scooping blade 120 and a pair of actuating levers 114 at either end of the scooping blade 120. Each arm of the pair of actuating levers 114 is preferably L-shaped, but other shapes are also suitable. The actuating levers 114 are connected to the scooping blade 120 at each side by rectangular or otherwise oblong joints having a thickness of at least that of the walls of the cylinder body 112. These rectangular or oblong pivot joints should be such that the scooping-blade assembly slides into the complementary installation channels 118 in a specific orientation but not at any other angle. For example, the rectangular joints depicted in FIGS. 1-4 prevent the scooping assembly from accidentally detaching from the bottom of the cylinder body. The rectangular joints are small enough to completely fit within their corresponding pivot seats, yet large enough so that they must be aligned precisely lengthwise (relative to the installation channels 118) to pass through the installation channels 118. Such an arrangement allows the practitioner to pivot the scooping-blade assembly about the pivot joints during operation without fear of accidental detachment, so long as the practitioner does not simultaneously lift the cylinder body 112 while the pivot joints are precisely aligned lengthwise with the installation channels 118. Because the scooping blade 120 only needs to rotate approximately 90 degrees during operation, the long axes of the pivot joints are preferably oriented at such an angle so that the long axes will never align with the installation channels 118 during normal use. Thus, the pivot joints (or oblong connectors) point at an angle between the actuating lever arms 114 and the scooping blade 120. The long axes of the pivot joints are preferably oriented about 45 degrees from the scooping blade's 120 plane of curvature.

The scooping blade 120 in the first embodiment should be beveled (or otherwise thicker along the center for strength) and sharp on both edges so that the scooping blade 120, when fully extended (i.e. the axis of curvature of the scooping blade is perpendicular to the cylinder body's axis), can carve through the dermis and lipid layers to be sampled. The diameter of the scooping blade 120 should be slightly less than the inner diameter of the cylinder body 112 such that the scooping blade 120 can pivot freely about its pivot joints when seated within the pivot seats and enclosed within the cylinder body 112.

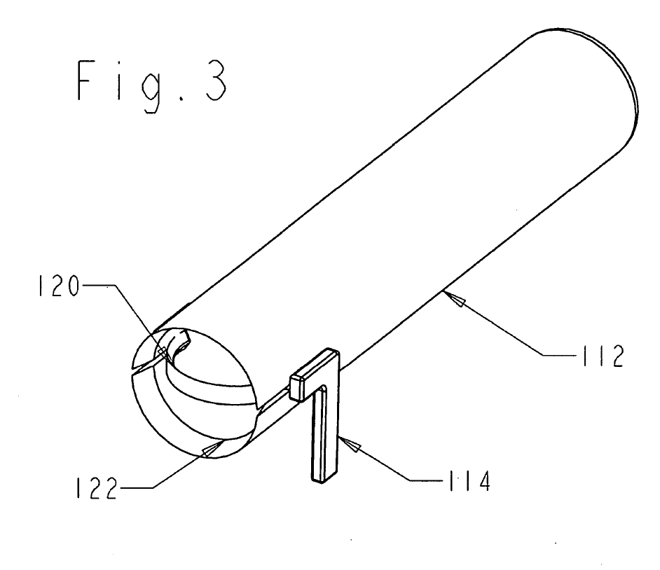

In practice, the selected area of the skin is cleaned with a sterilizing solution such as povidone-iodine solution and anesthetized using, for example, a lidocaine-with-epinephrine solution. The sterilized biopsy device of the present invention is assembled by aligning the pivot joints with the installation channels 118 and sliding the scooping-blade assembly up into the cylinder body 112 until the pivot joints reach the pivot seats 116. The scooping-blade assembly is then rotated slightly about its pivot joints held within the pivot seats 116 until the scooping blade 120 is flush with the inner surface of the cylinder body 112. Next, the entire biopsy device is held vertically over the portion of the skin to be sampled, and the device is simultaneously rotated and pushed downward to cause the biopsy device's coring blade 122 to penetrate the skin. Once the practitioner reaches the desired depth (typically breaking through the dermis and into the subcutaneous lipid layers), the actuating lever arms 114 are used to rotate the scooping blade 120 from a fully-retracted position (as shown in FIG. 1) to a fully-extended position (as shown in FIG. 3). This 90-degree rotation causes the scooping blade 120 to make a semi-spherical incision at the bottom of the cylinder body 112. Once fully extended, the scooping blade 120 is spun about the center axis of the cylinder body 112. This spinning of the cylinder body 112 and scooping blade 120 causes the scooping blade 120 to sculpt a hemispherical bottom for the cored skin sample, thereby detaching the biopsy sample from the lower layers prior to lifting and/or removing the sample. In the event that a simple, single 90-degree motion of the scooping blade 120 is too difficult to perform (which may happen due to the toughness of the sampling area, etc.), such rotation of the scooping blade 120 can be performed simultaneously with a continued spinning motion of the cylinder body 112, thereby easing the coring and detaching steps. Once the tissue has been cored and detached from the lower layers (by scooping the sample bottom), the entire biopsy device and the biopsy specimen can be lifted vertically and without further incision. In its fully-extended position, the scooping blade 120 also serves as a retaining cross-member for retaining the biopsy specimen within the cylinder body 112 during lifting and removal of the biopsy device. The actuating lever arms 114 can be returned to their starting positions to retract the scooping blade 120 and release the biopsy specimen.

As previously mentioned, FIGS. 5 through 8 depict a second, more preferable embodiment, which will be discussed below in further detail.

A second, more preferable embodiment of a biopsy device for assisting in the removal of a biopsy specimen comprises: a hollow cylinder body 212; a coring blade 222 at the base of the hollow cylinder body 212; at least one scooping blade 220 pivotally secured by an axle 218 and a pair of pivot seats 232 within the cylinder body 212, where each scooping blade 220 is semicircular; a longitudinally-moveable plunger 214 secured within the hollow cylinder 212, where the plunger 214 has at least one plunger leg 216 for contacting each scooping blade 220, and each plunger leg 216, via longitudinal plunger movement, is capable of applying torque to its respective scooping blade 220, thereby enabling rotational scooping-blade movement about said axle 218. As shown in FIGS. 5-9, the plunger 214 is moveably secured within, and in longitudinal alignment with, the hollow cylinder 212 via a plunger guide 224. Although the specific plunger guide 224 shown in the figures is a disc spanning the inner diameter of the cylinder body 212 and having a center hole for accommodating the shaft of the plunger 214, any plunger guide 224 can be substituted, as it is not essential to the present invention. Each plunger leg 216 can further comprise a plunger foot 230 at its lower end to ensure good contact between the plunger 214 and the scooping blades 220 when the plunger 214 is depressed. To improve ease of use, the device can further include a pair of finger grips 228 near the upper end of the cylinder body 212 and a plunger top 226 to provide a more comfortable, ergonomic interface for the operator's fingers and thumb.

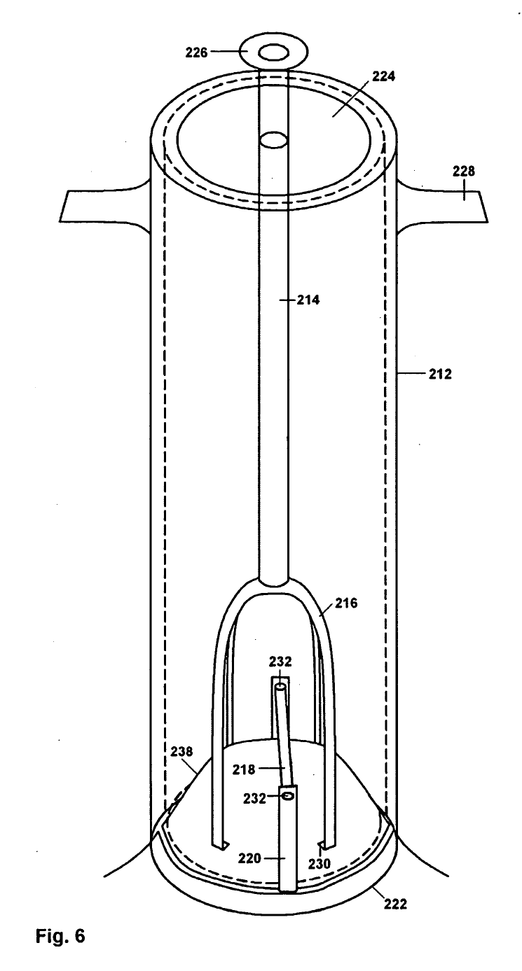

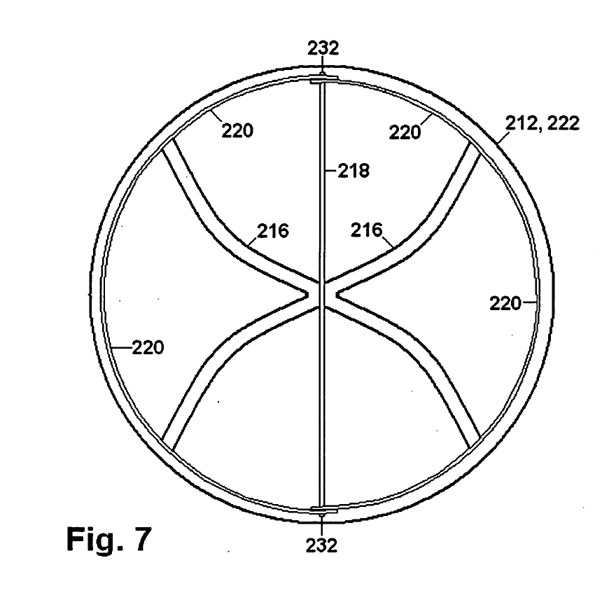

As can be seen in FIG. 7, both scooping blades 220 share the same axle 218, which is secured by two pivot seats 232 to the lower end of the cylinder body 212 near the coring blade 222. One of the scooping blades 220 has a slightly smaller diameter than the other so that when the plunger legs 216 push them downward, both blades can fully align and concentrically overlap each other, as shown in FIG. 6. Because the scooping blades 220 will have fully carved through and detached the biopsy sample 238 by the time both blades 220 meet and overlap (as shown in FIG. 6), the scooping blades 220 need only have one sharp edge (in the fully-retracted position as shown in FIG. 5, the sharp edge of each scooping blade 220 should be facing downward).

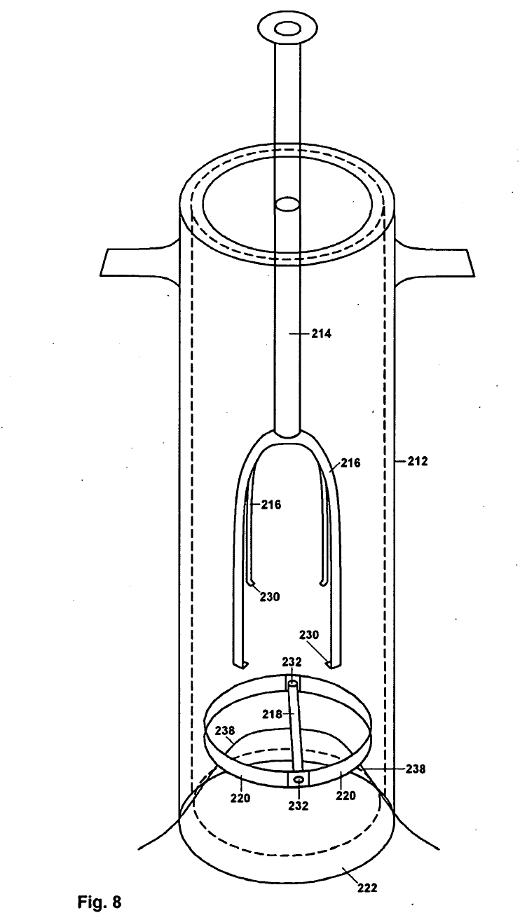

In practice, the scooping blades 220 begin in a retracted position as shown in FIG. 5 and/or FIG. 8. The plunger 214 can either rest atop the scooping blades 220, as shown in FIG. 5, or begin in an elevated position above the scooping blades 220, as shown in FIG. 8. An elevated starting orientation as shown in FIG. 8 may be particularly useful in assisted-actuation variations of the device—for example, where the plunger 214 begins under spring compression by a spring (not shown) compressed along the shaft of the plunger 214 between the plunger guide 224 and the plunger legs 216. The cylinder body 212 is initially pushed downward and rotated to enable the coring blade 222 to make a cylindrical incision around the biopsy area. Once the coring blade 222 has reached the desired depth, the plunger can then be activated to rotate the scooping blades 220 downward and about the axle 218, thereby severing the cored biopsy sample from the patient.

The plunger 214 can be activated in several ways to effect biopsy sample detachment. For example, the plunger 214 and its legs 216 can be pushed downward and rotated (less than one-quarter turn, in most cases) to force the scooping blades 220 together. Depending on the exact dimensions of the plunger legs 216 and the scooping blades 220 (the figures are not necessarily drawn to scale), the plunger may or may not require rotation in addition to downward motion to fully deploy the scooping blades 220. Where the particular dimensions of the plunger legs 216 and scooping blades 220 require some rotation of the plunger 214 to fully-deploy the scooping blades 220, the plunger 214 can either be rotated by hand, or rotation can be induced by rifling of the plunger shaft and/or the plunger guide 224. In circumstances where only a simple downward force is required (for example, where rifling is present), the downward force can be supplied either by hand or with assistance by spring, hammer, electricity, pneumatics, etc. In some situations, it is possible to impart enough momentum to the scooping blades 220 to force them together without maintaining plunger-to-blade contact throughout the entire downward rotation of the scooping blades 220.

Once the biopsy sample 238 is fully severed by the scooping blades, 220, the cylinder body 212 and contents (including the biopsy sample 238) can be withdrawn from the patient. The scooping blades 220 in their fully-deployed orientation help retain the biopsy sample 238 within the cylinder body 212 while it is being withdrawn. To remove the biopsy sample 238 from the cylinder body 212, the user simply retracts the plunger 214 and rotates the scooping blades 220 away from each other.

Note that while the scooping blades 220 are shown within the cylinder body 212 in the particular embodiment depicted in FIGS. 5-9, the scooping blades alternatively can be rotatably-secured to the outside of the cylinder body. The plunger mechanism might alternatively take the form of a moveable outer sleeve about the cylinder body, where the sleeve has downward-extending legs for contacting the scooping blades. The scooping blades would then be forced together by pushing the outer sleeve downward so that the sleeve's downward-extending legs push and rotate the scooping blades toward each other. If necessary, the outer sleeve might also be rotated to cause its downward-extending legs to force the scooping blades together. Again, activation of the plunger can be hand-operated or assisted.

While the invention has been particularly shown and described with reference to a preferred embodiment, it will be understood by those skilled in the art that various changes in form and detail may be made therein without departing from the spirit and scope of the invention.

We claim:

1. A biopsy device for assisting in the removal of a biopsy specimen, said biopsy device comprising:
a hollow cylinder body;
a coring blade at the base of the hollow cylinder body;
at least one scooping blade pivotally secured by an axle and a pair of pivot seats within the cylinder body, where each scooping blade is semicircular;
a longitudinally-moveable plunger secured within the hollow cylinder body, where the plunger has at least one plunger leg for contacting each scooping blade, and each plunger leg, via longitudinal plunger movement, is capable of applying torque to its respective scooping blade, thereby enabling rotational scooping-blade movement about said axle.

2. The biopsy device of claim 1 wherein said biopsy device is made of a medical-grade stainless steel.

3. The biopsy device of claim 1 wherein the inner diameter of the cylinder body is within the range of about 2 mm to about 5 mm.

4. The biopsy device of claim 1 wherein the inner diameter of the cylinder body is within the range of about 3 mm to about 4 mm.
